(19) United States
(12) Patent Application Publication
(10) Pub. No.: US 2007/0293582 A1
(43) Pub. Date: Dec. 20, 2007

(54) Epinephrine dosing regimens comprising buccal, lingual or sublingual and injectable dosage forms

(76) Inventor: Malcolm Hill, Solana Beach, CA (US)

Correspondence Address: WILSON SONSINI GOODRICH & ROSATI, 650 PAGE MILL ROAD, PALO ALTO, CA, 94304-1050, US

(21) Appl. No.: 11/810,697

(22) Filed: Jun. 5, 2007

Related U.S. Application Data
(60) Provisional application No. 60/803,968, filed on Jun. 5, 2006.
(60) Provisional application No. 60/803,975, filed on Jun. 5, 2006.
(60) Provisional application No. 60/807,181, filed on Jul. 12, 2006.

(51) Int. Cl. A61K 31/137

(52) U.S. Cl. 514/649

(57) ABSTRACT

The present invention relates to methods of administering a series of epinephrine doses for the treatment of allergic emergencies, including anaphylaxis, comprising buccal, lingual or sublingual epinephrine dosage forms and injectable epinephrine dosage forms. Also provided herein are kits and packaging systems useful in these methods.

### RELATED APPLICATIONS

This application claims the benefit of U.S. Provisional Application Nos. 60/803,968, filed Jun. 5, 2006; 60/803,975, filed Jun. 5, 2006; and 60/807,181, filed Jul. 12, 2006, which are hereby incorporated by reference in their entireties.

### FIELD OF INVENTION

The present invention relates to methods of administering a series of epinephrine doses for the treatment of allergic emergencies, including anaphylaxis, comprising buccal, lingual or sublingual epinephrine dosage forms and injectable epinephrine dosage forms. Also provided herein are kits and packaging systems useful in these methods.

### BACKGROUND OF THE INVENTION

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within about a minute to about two hours after contact with the allergy-causing substance; but in rare instances onset may be delayed by as much as eight hours. Contact with anaphylaxis-inducing agents, and the severity of the resulting anaphylactic reaction, can be extremely unpredictable. Accordingly, allergists recommend that persons who have a personal or family history of anaphylaxis, or a risk of anaphylaxis, be prepared to self-administer emergency treatment at all times. Additionally, adults charged with caring for children who are at risk for anaphylaxis should also be prepared to administer anti-anaphylactic first aid.

The symptoms of anaphylaxis include one or more of the following, generally within 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or bronchospasm, nausea, vomiting, abdominal pain, diarrhea, shock, convulsions, incontinence, unresponsiveness and death. An anaphylactic reaction may include cardiovascular collapse, even in the absence of respiratory symptoms.

According to the Merck Manual, immediate treatment with epinephrine is imperative for the successful treatment of anaphylaxis. Merck Manual, 17^{th} Ed., 1053-1054 (1999). The recommended dose of epinephrine for the treatment of anaphylaxis is about 0.01 mg/Kg: usually about 0.2 to about 0.5 mL of a 1:1000 dilution of epinephrine in a suitable carrier. It is further recommended that, if the symptoms of anaphylaxis persist after the first dose of epinephrine, the patient be treated with epinephrine doses every five minutes after the initial dose until there is resolution of the anaphylactic symptoms or until the onset of epinephrine toxicity. See Leiberman et al., (2005) J. Allergy Clin. Immunol. 115: S483-S523.

Typically, the epinephrine doses are given manually, either subcutaneously (SQ) or intra-muscularly (IM), and in recent years automatic injectors have become an accepted first aid means of delivering epinephrine. It is recommended that persons at risk of anaphylaxis, and persons responsible for children at risk for anaphylaxis, maintain one or more automatic epinephrine injectors in a convenient place at all times.

Given the potential difficulties associated with the administration of multiple sequential subcutaneous or intra-muscular administrations of epinephrine, such as patient apprehension or the need to always maintain multiple epinephrine injectors close at hand, there exists a need in the art for additional methods of immediately administering epinephrine to a person undergoing anaphylaxis wherein the epinephrine can be administered in a series of sequential dosage forms which do not rely solely on epinephrine injectors. There also exists a need in the art for methods of immediately administering epinephrine which provide alternate routes of administration of the epinephrine within the same dosing regimen. For example, if a patient has been administered an injectable dosage form comprising epinephrine but the injectable dosage form was not effective or well tolerated, such a patient may need to continue epinephrine therapy via an alternate route of administration, e.g., via buccal, lingual, or sublingual delivery. There is also a need for kits or packaging systems comprising a series of epinephrine dosage forms wherein not all of the dosage forms are injectable and wherein the dosage forms are enclosed in a package having markings or instructions for use in the treatment of anaphylaxis.

The present invention meets the foregoing needs and provides related advantages as well.

### SUMMARY OF THE INVENTION

The present invention meets the foregoing and related needs by providing an improved method of treating allergic emergencies, including anaphylaxis, with epinephrine in patients where current treatments are not ideal.

Provided herein are methods for treating an allergic emergency in a patient comprising the steps of (a) administering to the patient at least one dose of a buccal, lingual or sublingual dosage form comprising epinephrine; and (b) administering to the patient at least one dose of an injectable dosage form comprising epinephrine. In certain embodiments, the methods comprise first administering to said patient at least one dose of a buccal, lingual or sublingual dosage form comprising epinephrine and subsequently administering to said patient at least one dose of an injectable dosage form comprising epinephrine. In certain other embodiments, the methods comprise first administering to said patient at least one dose of an injectable dosage form comprising epinephrine and subsequently administering to said patient at least one dose of a buccal, lingual or sublingual dosage form comprising epinephrine.

In other embodiments, the present invention provides methods for treating an allergic emergency in a patient comprising the steps of (a) administering to the patient one dose of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine. In another embodiment, the patient is administered a first and second dose of an injectable dosage form comprising epinephrine. In still another embodiment, the patient is administered a first, second, and third dose of an injectable dosage form comprising epinephrine.

In certain embodiments, the present invention provides methods for treating an allergic emergency in a patient comprising the steps of (a) administering to the patient two doses of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine. In another embodiment, the patient is administered a first and second dose of an injectable dosage form comprising epinephrine. In still another embodiment, the patient is administered a first, second, and third dose of an injectable dosage form comprising epinephrine.

In certain embodiments, the present invention provides methods for treating an allergic emergency in a patient comprising the steps of (a) administering to the patient two doses of a buccal, lingual or sublingual dosage form comprising epinephrine; and (b) administering to the patient a first dose of an injectable dosage form comprising epinephrine.

In certain other embodiments, the present invention provides methods for treating an allergic emergency in a patient comprising the steps of (a) administering to the patient three doses of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine: In another embodiment, the patient is administered a first and second dose of an injectable dosage form comprising epinephrine. In still another embodiment, the patient is administered a first, second, and third dose of an injectable dosage form comprising epinephrine.

In other embodiments, the present invention provides methods for treating an allergic emergency in a patient comprising the steps of (a) administering to the patient one dose of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain other embodiments, the present invention provides methods for treating an allergic emergency in a patient comprising the steps of (a) administering to the patient two doses of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain other embodiments, the present invention provides methods for treating an allergic emergency in a patient comprising the steps of (a) administering to the patient two doses of an injectable dosage form comprising epinephrine; and (b) administering to the patient a first dose of a buccal, lingual or sublingual dosage form comprising epinephrine.

In certain other embodiments, the present invention provides methods for treating an allergic emergency in a patient comprising the steps of (a) administering to the patient three doses of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain embodiments, the each buccal, lingual or sublingual dosage form comprises an amount of epinephrine that is bioequivalent to about 0.01 mg/Kg of epinephrine administered by intra-muscular administration. In other embodiments, each buccal, lingual or sublingual dosage form comprises an amount of epinephrine that is bioequivalent to about 0.10 mg to about 0.50 mg of epinephrine administered by intra-muscular injection. In one embodiment, each buccal, lingual or sublingual dosage form comprises an amount of epinephrine that is bioequivalent to about 0.10 mg of epinephrine administered by intra-muscular injection. In another embodiment, each buccal, lingual or sublingual dosage form comprises an amount of epinephrine that is bioequivalent to about 0.15 mg of epinephrine administered by intra-muscular injection. In still another embodiment, each buccal, lingual or sublingual dosage form comprises an amount of epinephrine that is bioequivalent to about 0.30 mg of epinephrine administered by intra-muscular injection. In yet another embodiment, each buccal, lingual or sublingual dosage form comprises an amount of epinephrine that is bioequivalent to about 0.45 mg of epinephrine administered by intramuscular injection. In still yet another embodiment, each buccal, lingual or sublingual dosage form comprises an amount of epinephrine that is bioequivalent to about 0.50 mg of epinephrine administered by intra-muscular injection. In yet other embodiments, each buccal, lingual or sublingual dosage form comprises from about 1 mg to about 100 mg of epinephrine. In still other embodiments, the buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intra-muscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intra-muscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intra-muscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In some embodiments, the buccal, lingual or sublingual dosage forms can be tablets. In certain embodiments, the dosage form is a lingual tablet. In certain other embodiments, the dosage form is a sublingual tablet. In other embodiments, the dosage form is a buccal tablet. In some embodiments, the buccal, lingual or sublingual dosage forms further comprise a pharmaceutically acceptable excipient.

In other embodiments, each injectable dose of epinephrine solution can comprise from about 0.1 mg to about 0.6 mg of epinephrine. In still other embodiments, each injectable dose of epinephrine solution can comprise from about 0.2 mg to about 0.5 mg of epinephrine. In one embodiment, each injectable dose of epinephrine solution comprises about 0.15 mg of epinephrine. In another embodiment, each injectable dose of epinephrine solution comprises about 0.3 mg of epinephrine. In yet another embodiment, each injectable dose of epinephrine solution comprises about 0.5 mg of epinephrine.

In still other embodiments, each injectable dose of epinephrine solution can comprise from about 0.25 mg/mL to about 5.0 mg/mL of epinephrine. In still other embodiments, each injectable dose of epinephrine solution can comprise from about 0.5 mg/mL to about 3.0 mg/mL of epinephrine. In yet other embodiments, each injectable dose of epinephrine solution can comprise about 0.5 mg/mL to about 1.5 mg/mL of epinephrine. In one embodiment, each injectable dose of epinephrine solution comprises about 1.0 mg/mL of epinephrine.

In some embodiments, each injectable dose of epinephrine solution further comprises at least one pharmaceutically inactive ingredient.

In certain embodiments, the injectable dosage forms are automatically injected. In other embodiments, the injectable dosage forms are manually injected.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the second or more dose of a buccal, lingual or sublingual dosage form comprising epinephrine comprises an amount of epinephrine greater than the first dose of a buccal, lingual or sublingual dosage form comprising epinephrine. In other embodiments, the second or more dose of a buccal, lingual or sublingual dosage form comprising epinephrine comprises an amount of epinephrine less than the first dose of a buccal, lingual or sublingual dosage form comprising epinephrine. In still other embodiments, the second or more dose of a buccal, lingual or sublingual dosage form comprising epinephrine comprises an amount of epinephrine that is from about 25% to about 200% of the amount of epinephrine in the first dose of a buccal, lingual or sublingual dosage form comprising epinephrine.

In still other embodiments, the second or more dose of an injectable dosage form comprising epinephrine comprises an amount of epinephrine greater than the first dose of an injectable dosage form comprising epinephrine. In other embodiments, the second or more dose of an injectable dosage form comprising epinephrine comprises an amount of epinephrine less than the first dose of an injectable dosage form comprising epinephrine. In still other embodiments, the second or more dose of an injectable dosage form comprising epinephrine comprises an amount of epinephrine that is from about 25% to about 200% of the amount of epinephrine in the first dose of an injectable dosage form comprising epinephrine.

In some aspects of the present invention, the time interval between consecutive or sequential doses can be the amount of time it takes to see a therapeutic effect in the patient. In some embodiments, the methods comprise a time interval between administrations of each consecutive or sequential dose of between about 3 minutes to about 10 minutes. In other embodiments, the time interval between consecutive or sequential administrations is about 5 minutes.

The present invention further provides a kit or packaging system for treating an allergic emergency in a patient, comprising (a) at least one dose of a buccal, lingual or sublingual dosage form comprising epinephrine; and (b) at least one dose of an injectable dosage form comprising epinephrine. In some embodiments, the kit or packaging system further comprises written instructions for administering the at least one dose of a buccal, lingual or sublingual dosage form comprising epinephrine and the at least one dose of injectable dosage form comprising epinephrine.

In certain embodiments, the written instructions provide that (a) the at least one dose of a buccal, lingual or sublingual dosage form comprising epinephrine is first administered to said patient to provide a therapeutic effect in response to said allergic emergency; and/or optionally (b) subsequent to the first administration of the at least one dose of a buccal, lingual or sublingual dosage form comprising epinephrine, the at least one dose of an injectable dosage form comprising epinephrine is administered to said patient.

In certain other embodiments, the written instructions provide that (a) the at least one dose of an injectable dosage form comprising epinephrine is first administered to said patient to provide a therapeutic effect in response to said allergic emergency; and/or optionally (b) subsequent to the first administration of the at least one dose of an injectable dosage form comprising epinephrine, the at least one dose of a buccal, lingual or sublingual dosage form comprising epinephrine is administered to said patient.

In some embodiments, the at least one dose of a buccal, lingual or sublingual dosage form comprising epinephrine is contained within a protective liner. In one embodiment, the protective liner prevents damage due to moisture, light, or oxygen. In another embodiment, the protective liner is a polymer-lined foil. In yet another embodiment, the doses are identified in the kit or packaging system. In still another embodiment, the doses are identified by numerical markings or by location within the kit or packaging system. In yet still another embodiment, the identification of the doses indicates the order in which the doses are administered to the patient. In other embodiments, the kit or packaging system further comprises a carrying case.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1: FIG. 1 provides an illustration of one embodiment of a kit or packaging system as described herein which comprises: (a) five (5) sublingual dosage forms comprising epinephrine housed in a blister package which is peelably secured to top flap of the packaging system, wherein each sublingual dosage form is identified by numerical marking embossed onto the blister package and (b) an automatic-manual injector device comprising an epinephrine solution.

FIG. 2: FIG. 2 provides a cross-sectional view of one embodiment of a kit comprising a carrying case as described here which comprises: (a) three (3) sublingual dosage forms comprising epinephrine housed within the top of the carrying case, each of which is individually contained in a blister package and (b) an automatic-manual injector device comprising an epinephrine solution. The figure further illustrates the hinged top of the carrying case which is open to expose the sublingual dosage forms housed within the top of the carrying case.

### DETAILED DESCRIPTION OF THE INVENTION

The present invention provides methods for treating allergic emergencies, such as anaphylaxis. The invention further provides dosing regimens of epinephrine for treating allergic emergencies, such as anaphylaxis, comprising the administration of at least one dose of a buccal, lingual or sublingual dosage form comprising epinephrine and at least one dose of epinephrine in an injectable dosage form. Furthermore, the invention provides kits or packaging systems comprising buccal, lingual or sublingual dosage forms of epinephrine in combination with one or more injectable epinephrine dosage forms for treating allergic emergencies, such as anaphylaxis.

As used herein, the term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, are intended to be included within the cited values.

As used herein, the terms “comprising,” “including,” “such as,” and “for example” are used in their open, non-limiting sense.

As used herein, “bioequivalent” or “bioequivalency” refers to one type of dosage form of a certain dose, e.g., a buccal, lingual or sublingual dosage form comprising about 1 mg to about 100 mg of epinephrine, having the same rate and extent of drug delivery as another type of dosage form at a certain dose, e.g., an intra-muscular injection of 0.3 mg of epinephrine. Bioequivalency can be shown by any method known in the art of pharmacodynamics or pharmacokinetics, and includes, but is not limited to, studies demonstrating that there is no significant difference between one type of dosage form and another type of dosage form for the mean maximal drug concentration (Cₘₐₓ), the area under the drug concentration time curve (AUC), or the time to maximum concentration in the blood (Tₘₐₓ). In certain aspects of the present invention, bioequivalency can be established by studies which demonstrate that there is no significant difference between one type of dosage form and another type of dosage form with regard to the mean maximal drug concentration (Cₘₐₓ) and the area under the drug concentration time curve (AUC). In certain other aspects of the present invention, bioequivalency can be established by studies which demonstrate that there is no significant difference between one type of dosage form and another type of dosage form with regard any one parameter of pharmacodynamics or pharmacokinetics, including, but not limited to, the mean maximal drug concentration (Cₘₐₓ), the area under the drug concentration time curve (AUC), or the time to maximum concentration in the blood (Tₘₐₓ).

“Therapeutic effect,” as used herein, refers to the amelioration, prevention, inhibition, relief, or termination of any of the symptoms of an allergic emergency described herein.

As used herein, “transmucosal drug delivery,” refers to the delivery of a pharmaceutically active agent through the epithelium for either local or systemic treatment. In some embodiments, transmucosal drug delivery comprises buccal delivery of epinephrine. In certain embodiments, the transmucosal drug delivery comprises lingual delivery of epinephrine. In certain other embodiments, transmucosal drug delivery comprises sublingual delivery of epinephrine. In still other embodiments, transmucosal drug delivery comprises rectal delivery of epinephrine.

As used herein, “buccal dosage forms,” refer to dosage forms which provide transmucosal delivery of an active agent, e.g., epinephrine, primarily through the epithelial cells of the oral cavity, e.g., the cheek. Buccal dosage forms are known in the art and can include, but are not limited to, patches, lozenges, tablets, oral dissolving/disintegrating tablets (ODTs), muco-adhesive tablets (including muco-adhesive films), fast-melt dissolving tablets (including fast-dissolving films), and the like.

As used herein, “lingual dosage forms,” which provide transmucosal delivery of an active agent, e.g., epinephrine, primarily through the oral epithelium. Lingual dosage forms are known in the art and can include, but are not limited to, lozenges, tablets, oral dissolving/disintegrating tablets (ODTs), fast-melt dissolving tablets (including fast-melt dissolving films), orally disintegrating dosage forms, troches, and the like.

As used herein, “sublingual dosage forms,” refer to dosage forms which provide transmucosal delivery of an active agent, e.g., epinephrine, primarily through the oral epithelium beneath the tongue. Sublingual dosage forms are known in the art and can include, but are not limited to, lozenges, tablets, oral dissolving/disintegrating tablets (ODTs), muco-adhesive tablets (including muco-adhesive films), fast-melt dissolving tablets (including fast-melt dissolving films), orally disintegrating dosage forms, troches, and the like.

As used herein, buccal, lingual and sublingual dosage forms refer to oral dosage forms wherein the primary route of administration for the active agent is via the epithelial lining of the oral cavity, e.g., the epithelial cells of the cheek or beneath the tongue. However, it is to be understood that small amounts of active agents delivered by such dosage forms may be swallowed by the patient and absorbed outside the oral cavity.

As used herein, “rectal dosage forms,” refer to dosage forms which provide transmucosal delivery of an active agent, e.g., epinephrine, primarily through the epithelial cells of the rectal cavity. Rectal dosage forms are known in the art and can include, but are not limited to, suppositories, rectal capsules, gels, creams, ointments, and the like.

As used herein, “injectable dosage forms” refer to dosage forms wherein an active agent, e.g., epinephrine, is delivered via an injector, e.g. a device comprising a needle, which provides for the subcutaneous or intra-muscular delivery of the active agent. Injectable dosage forms are known in the art and can include, but are not limited to, single dose manual or automatic devices, multiple dose manual-manual devices, multiple dose automatic-automatic devices, multiple dose manual-automatic devices, and multiple dose automatic-manual devices.

As described above, anaphylaxis means an acute and severe allergic reaction to an allergen or antigen. Treatment of anaphylaxis means at least partially ameliorating or alleviating the symptoms of anaphylaxis. Such treatment may be, and in most cases is, temporary. For example, in certain embodiments of the present invention, the methods, dosing regimens, and kits or packaging systems comprising buccal, lingual or sublingual dosage forms of epinephrine in combination with one or more injectable epinephrine dosage forms will provide emergency relief from the symptoms of anaphylaxis for a time sufficient for the patient to seek professional medical assistance. Thus, the methods, dosing regimens and kits or packaging systems of the invention are well suited for inclusion in first aid kits in professional child care settings and homes, especially where one or more persons at risk for anaphylaxis are known to dwell. They are also well suited for inclusion in so-called crash carts in ambulances or other emergency vehicles, as well as medical emergency rooms. They may also be conveniently carried by those who are at risk for anaphylaxis or those who are charged with caring for those who are at risk for anaphylaxis. The methods of the invention are suitable for treating persons who are at risk for allergic emergencies, such as anaphylaxis, in any of the aforementioned settings.

Thus, treatment of an allergic emergency includes treatment of anaphylaxis, for which the invention is especially well-suited. In addition, treatment of allergic emergency includes treatment of other allergic conditions that may be treated with epinephrine. For example, the symptoms of anaphylactoid reactions to drugs closely mimic those of anaphylaxis and are treated in a similar manner. In cases where it is not clear whether the reaction is a systemic immunological response (anaphylaxis) or a systemic toxic response (anaphylactoid reaction), the accepted first line of treatment is with epinephrine. In this sense, treatment of an allergic emergency encompasses the treatment of anaphylaxis, the treatment of anaphylactoid reaction, or both. See Leiberman et al., (2005) J. Allergy Clin. Immunol. 115: S483-S523.

Buccal, Lingual and Sublingual Epinephrine Dosage Forms

The present invention provides methods of treating an allergic emergency, such as anaphylaxis, in a patient, comprising administering to the patient at least one buccal, lingual or sublingual dosage form comprising epinephrine in combination with at least one injectable epinephrine dosage form comprising epinephrine. The methods described herein can be practiced using any pharmaceutical composition or dosage form containing epinephrine that is appropriate for buccal, lingual or sublingual administration. The discrete dosage forms of the present invention can comprise dosages of from about 1 mg to about 100 mg, and in some embodiments from about 15 mg to about 60 mg, of epinephrine. It is to be understood that epinephrine, as used herein, refers to both the free base form as well as any suitable pharmaceutically acceptable salt of epinephrine including, but not limited to, epinephrine bitartrate or epinephrine HCl salt. In certain embodiments, the methods of the present invention can include the use of a buccal, lingual or sublingual dosage form such as a disintegrating or dissolving tablet (ODTs) formulated for immediate disintegration or dissolution in the patient's mouth. In such embodiments, the buccal, lingual or sublingual tablet can disintegrate or dissolve without extracorporeal water. Thus, the saliva present in the patient's mouth is sufficient to initiate disintegration or dissolution of the sublingual tablet in the oral cavity. In such an embodiment, the epinephrine can be absorbed much more quickly than traditional oral dosage forms and can provide a rapid onset of epinephrine activity via absorption into the systemic circulation.

Ingredients and exemplary buccal, lingual or sublingual formulations can be found in Remington: The Science and Practice of Pharmacy 20th ed. (Lippincott Williams & Wilkins, 2000). The patent literature also contains many disclosures of buccal, lingual and sublingual formulations, including U.S. Pat. Nos. 7,067,116; 7,025,983; 6,923,981; 6,596,298; 6,726,928; 6,709,669; 6,509,040; 6,413,549; 5,976,577; 5,827,541; 5,738,875; 5,648,093; 5,631,023; 5,188,825; 4,020,558; 4,229,447; 3,972,995; 3,870,790; 3,444,858; 2,698,822; 3,632,743, and U.S. Published Application Nos. 20070059361; 20040247648; 20040131661; and 20040028730, each of which is specifically incorporated herein by reference in their entirety. In some embodiments, the dosage forms are prepared using pharmaceutically acceptable excipients. The excipients are known to those skilled in the preparation of buccal, lingual or sublingual dosage forms. For example, excipients that are commonly formulated into buccal, lingual and sublingual dosage forms include maltodextrin, colloidal silicon dioxide, starch, starch syrup, sugar and α-lactose.

In certain embodiments of the buccal, lingual or sublingual dosage forms described herein, excipients acting as disintegrants or dissolution enhancing agents can be incorporated into the formulation to provide for faster tablet disintegration or dissolution. In other embodiments, the buccal, lingual or sublingual epinephrine tablets can be formulated using absorption enhancers to maximize the release rate of the epinephrine into to the systemic circulation. In yet other embodiments, the absorption enhancer is a transmucosal absorption enhancer. Transmucosal absorption enhancers are known in the art and include, but are not limited to, chelators (e.g., EDTA, EGTA), non-ionic surfactants (e.g., 23-lauryl ether, laureth-9, polysorbates (including polysorbate 80), sucrose esters, or dodecylmaltoside), cationic surfactants (e.g., benzalkonium chloride or cetylmethylammonium bromide), anionic surfactants (e.g., sodium dodecyl glycocholate or sodium lauryl sulfate), bile salts and other steroidal detergents (e.g., cholate, deoxycholate, taurocholate, sodium glycocholate, sodium taurocholate, saponins, sodium taurodihydrofusidate or sodium glycodihydrofusidate), fatty acids (e.g., oleic acid, lauric acid capric acid, heptanoic acid, stearic acid, sucrose laurate, isopropyl myristate, sodium myristate or caprylic acid), and non-surfactants (e.g., aprotinin, dextran sulfate, sulfoxides, salicylates, Intravail® or 1-dodecylazacycloheptane-2-one (Azone)), phospholipids (e.g., phosphatidylcholines, lysophosphatidylcholine, or monoooleoyl phosphaltidyl ethanomamine), cyclodextrins, and various alkyl glycosides. See, e.g., Shojaei, 1998, J. Pharm. Pharmaceut. Sci. 1:15-30; and Mitra et al., 2002, Encyclopedia of Pharmaceutical Technology pp. 2081-2095. In certain embodiments, the transmucosal absorption enhancer can be Intravail® (Aegis Therapeutics, LLC, San Diego, Calif.). In other embodiments, the transmucosal absorption enhancer can be benzalkonium chloride.

In other embodiments, the active components of the epinephrine dosage forms described herein can further comprise other non-essential or less essential components or excipients known in the art, for example, but by no means limited to diluents, binders, glidants, lubricants, colorants, flavorants, coating materials and the like.

Diluents increase bulk of the composition to facilitate compression of the dosage form. As used herein, diluents include, but are not limited to, compounds such as lactose, starch, mannitol, sorbitol, dextrose, tricalcium phosphate, calcium phosphate; anhydrous lactose, spray-dried lactose; pregelatinized starch, compressible sugar, such as Di-Pac® (Amstar), hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride, and the like.

Binders, as used herein, refer to compounds which impart cohesive qualities to the formulation and include, but are not limited to, compounds such as alginic acid and salts thereof, cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

Lubricants and glidants are compounds that prevent, reduce or inhibit adhesion or friction of materials. Exemplary lubricants or glidants include, but are not limited to, stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

Flavoring agents and/or sweeteners useful in the epinephrine formulations described herein, include, but are not limited to, compounds such as acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

It should be appreciated that there is considerable overlap between excipients used in the solid dosage forms described herein. Thus, the above-listed excipients should be taken as merely exemplary, and not limiting, of the types of excipients that can be included in the buccal, lingual or sublingual dosage forms of the present invention. The amounts of such excipients can be readily determined by one skilled in the art, according to the particular properties desired.

Methods of Manufacturing Buccal, Lingual and Sublingual Dosage Forms Comprising Epinephrine

In addition, conventional methods of processing active ingredients and excipients into pharmaceutical compositions and dosage forms for buccal, lingual and sublingual administration are well known to the skilled formulation specialist. For example, various techniques are known in the art that can be used to formulate disintegrating or dissolving sublingual tablet dosage forms. Manufacturing processes for buccal, lingual and sublingual disintegrating tablets are known in the art and include, but are not limited to, conventional tableting techniques, freeze-dried technology, and floss-based tableting technology.

i. Conventional Techniques

Conventional tablet processing features conventional tablet characteristics for ease of handling, packaging, and fast disintegration (T. K. Ghosh, Oct. 29, 2003, American Association of Pharmaceutical Scientists). The technology is based on a combination of physically modified polysaccharides that have water dissolution characteristics that facilitate fast disintegration and high compressibility. The result is a fast-disintegrating tablet that has adequate hardness for packaging in bottles and easy handling.

In certain embodiments, the manufacturing process involves granulating low-moldable sugars (e.g., mannitol, lactose, glucose, sucrose, and erythritol) that show quick dissolution characteristics with high-moldable sugars (e.g., maltose, sorbitol, trehalose, and maltitol). The result is a mixture of excipients that have fast-dissolving and highly moldable characteristics (Hamilton et al., 2005, Drug Deliv. Technol. 5: 34-37). The epinephrine can be added, along with other standard tableting excipients, during the granulation or blending processes. The tablets are manufactured at a low compression force followed by an optional humidity conditioning treatment to increase tablet hardness (Parakh et al., 2003, Pharm. Tech. 27: 92-100).

In other embodiments, a compressed buccal, lingual or sublingual tablet comprising epinephrine is based on a conventional tableting process involving the direct compression of active ingredients, effervescent excipients, and taste-masking agents (see U.S. Pat. No. 5,223,614, which is herein incorporated by reference in its entirety). The tablet quickly disintegrates because effervescent carbon dioxide is produced upon contact with moisture. The effervescent excipient (known as effervescence couple) is prepared by coating the organic acid crystals using a stoichiometrically lesser amount of base material. The particle size of the organic acid crystals is carefully chosen to be larger than the base excipient to ensure uniform coating of the base excipient onto the acid crystals. The coating process is initiated by the addition of a reaction initiator, which is purified water in this case. The reaction is allowed to proceed only to the extent of completing the base coating on organic acid crystals. The required end-point for reaction termination is determined by measuring carbon dioxide evolution. Then, the excipient is mixed with the active ingredient or active microparticles and with other standard tableting excipients and then compressed into tablets.

In still other embodiments, the buccal, lingual or sublingual tablets are made by combining non-compressible fillers with a taste-masking excipient and active ingredient into a dry blend. The blend is compressed into tablets using a conventional rotary tablet press. Tablets made with this process have higher mechanical strength and are sufficiently robust to be packaged in blister packs or bottles (Aurora et al., 2005, Drug Deliv. Technol. 5:50-54). In other embodiments, the method further incorporates taste-masking sweeteners and flavoring agents such as mint, cherry, and orange. In certain embodiments, epinephrine tablets made with this process should disintegrate in the mouth in 5-45 seconds and can be formulated to be bioequivalent to intra-muscular or subcutaneous dosage forms containing epinephrine.

ii Freeze-Dried Buccal, Lingual or Sublingual Tablets Comprising Epinephrine

The freeze-drying process involves the removal of water (by sublimation upon freeze drying) from the liquid mixture of a drug (e.g., epinephrine), matrix former, and other excipients filled into preformed blister pockets. The formed matrix structure is very porous in nature and rapidly dissolves or disintegrates upon contact with saliva (Sastry el a., 2005, Drug Delivery to the Oral Cavity: Molecule to Market, pp. 311-316).

Common matrix-forming agents include gelatins, dextrans, or alginates which form glassy amorphous mixtures for providing structural strength; saccharides such as mannitol or sorbitol for imparting crystallinity and hardness; and water, which functions as a manufacturing process medium during the freeze-drying step to induce the porous structure upon sublimation. In addition, the matrix may contain taste-masking agents such as sweeteners, flavorants, pH-adjusting agents such as citric acid, and preservatives to ensure the aqueous stability of the suspended drug in media before sublimation.

In this embodiment, Freeze-dried buccal, lingual or sublingual ODTs comprising epinephrine can be manufactured and packaged in polyvinyl chloride or polyvinylidene chloride plastic packs, or they may be packed into laminates or aluminum multilaminate foil pouches to protect the product from external moisture.

Other known methods for manufacturing buccal, lingual or sublingual ODTs include lyophilization (e.g., Lyoc (Farmalyoc, now Cephalon, Franzer, Pa.) and QuickSolv (Janssen Pharmaceutica, Beerse, Belgium). Lyoc is a porous, solid wafer manufactured by lyophilizing an oil-in-water emulsion placed directly in a blister and subsequently sealed. The wafer can accommodate high drug dosing and disintegrates rapidly but has poor mechanical strength (see EP 0159237). QuickSolv tablets are made with a similar technology that creates a porous solid matrix by freezing an aqueous dispersion or solution of the matrix formulation. The process works by removing water using an excess of alcohol (solvent extraction). In certain embodiments, the manufacturing methods which utilize the lyophilization techniques, such as those related to QuickSolv as described above, could be of particular importance for producing buccal, lingual or sublingual ODTs comprising epinephrine. This is especially so in light of the data provided herein which shows the potential negative effect that highly water soluble excipients can have in the absorption of epinephrine in vivo. Thus, a buccal, lingual or sublingual ODT comprising epinephrine manufactured by such a lyophilization technique could provide increased in vivo epinephrine absorption due of the removal of water soluble excipients occurring during the water removal step as described above.

iii. Floss-Based Buccal, Lingual or Sublingual Tablets Comprising Epinephrine

In other embodiments, floss-based tablet technology (e.g., FlashDose, Biovail, Mississauga, ON, Canada) can be used to produce fast-dissolving buccal, lingual or sublingual tablets comprising epinephrine using a floss known as the shearform matrix. This floss is commonly composed of saccharides such as sucrose, dextrose, lactose, and fructose. The saccharides are converted into floss by the simultaneous action of flash-melting and centrifugal force in a heat-processing machine similar to that used to make cotton candy. See U.S. Pat. Nos. 5,587,172, 5,622,717, 5,567,439, 5,871,781, 5,654,003, and 5,622,716, each of which is specifically incorporated by reference herein in their entirety. The fibers produced are usually amorphous in nature and are partially re-crystallized, which results in a free-flowing floss. The floss can be mixed with epinephrine and pharmaceutically acceptable excipients followed by compression into a tablet that has fast-dissolving characteristics.

iv. Additional Methods of Formulating Buccal, Lingual or Sublingual Dosage Forms Comprising Epinephrine

Additional techniques can also be used to formulate the rapidly disintegrating or dissolving buccal, lingual or sublingual dosage forms of the present invention. See, Sastry et al., 2000, Pharm Sci. Technol. Today 3: 138-145; Chang et al. 2000, Pharm. Technol. 24: 52-58; Sharma et al., 2003 Pharm. Technol. of North America 10-15; and Allen, 2003, Int'l J. of Pharm. Technol. 7: 449-450, each of which is specifically incorporated herein by reference in their entirety. In some embodiments, direct compression can be used to formulate the buccal, lingual or sublingual epinephrine dosage forms of the present invention.

Other techniques useful in formulating the buccal, lingual, or sublingual dosage forms described herein include the formulation of rapidly dissolving oral films. These techniques are known in the art and described in, for example, U.S. Pat. Nos. 7,067,116; 7,025,983; 6,923,981; 6,596,298; and U.S. Published Application No. 20040247648, each of which is specifically incorporated herein in their entirety. In such embodiments, in addition to epinephrine, the rapidly dissolving oral films can comprise a film-forming agent, and at least one of the following additional ingredients: water, antimicrobial agents, plasticizing agents, flavoring agents, saliva stimulating agents, cooling agents, surfactants, stabilizing agents, emulsifying agents, thickening agents, binding agents, coloring agents, sweeteners, fragrances, triglycerides, preservatives, polyethylene oxides, propylene glycol, and the like. By way of a non-limiting example, the buccal, lingual, or sublingual rapidly dissolving oral films described herein can comprise a film-forming agent selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. In certain aspects, the rapidly dissolving films can further comprise a taste-masking agent, e.g., an ion exchange resin. In certain embodiments, the ion exchange resins for use in the dissolving films of the present invention are water-insoluble and consist of a pharmacologically inert organic or inorganic matrix containing covalently bound functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound ionic groups may be strongly acidic (e.g., sulfonic acid), weakly acidic (e.g., carboxylic acid), strongly basic (e.g., quaternary ammonium), weakly basic (e.g., primary amine), or a combination of acidic and basic groups. In still other aspects, the rapidly dissolving films can comprise modified starches which can significantly improve the overall stability and resistance of the film to adverse factors including heat and moisture for better product performance and improved storage life. Modified starches can also enable the dissolution of more solids (up to twice the amount attainable with unmodified starch) in the buccal, lingual, or sublingual film. In certain embodiments, the modified starches include modified corn starches, modified tapioca starches, acid and enzyme hydrolyzed corn and/or potato starches, hypochlorite-oxidized starches, acid-thinned starches, ethylated starches, cross-bonded starches, hydroxypropylated tapioca starches, hydroxypropylated corn starches, pregelatinized modified starches, and the like.

Still other techniques useful in formulating the buccal, lingual, or sublingual dosage forms described herein include the formulation of rapidly disintegrating or fast dispersing dosage forms which release the epinephrine rapidly on contact with a fluid (e.g., saliva, bodily fluids, water, and the like). These techniques are known in the art and are set forth in, for example, U.S. Pat. Nos. 6,726,928; 6,709,669; 5,976,577; 5,827,541; 5,738,875; 5,631,023; and 5,188,825, each of which is specifically incorporated herein by reference in their entirety. In certain aspects, such methods include the preparation freeze-dried dosage forms comprising epinephrine, wherein the epinephrine is bonded to an ion exchange resin to form a substantially water insoluble complex. This complex is then mixed with a compatible carrier and freeze-dried. In other aspects, such methods include preparation of an oral solid rapidly disintegrating dosage form comprising epinephrine comprising the formation of an aqueous solution and a suspension in an aqueous medium of an uncoated and uncomplexed epinephrine free base together with a carrier material selected from the group consisting of water-soluble and water-dispersible carrier materials and a compound which converts the epinephrine, which is present in its salt form, into its free base form and removing the aqueous medium. In still other aspects, such methods include buccal, lingual or sublingual dosage forms comprising epinephrine further comprising a carrier, wherein the carrier is gelatin and the dosage form is a fast-dispersing dosage form which releases the active ingredient rapidly on contact with a fluid (e.g., saliva or bodily fluids). In certain embodiments, the gelatin is a mammalian-derived gelatin. In other embodiments, the gelatin is a non-mammalian derived gelatin, such as fish gelatin.

Irrespective of the technique used to formulate the buccal, lingual or sublingual epinephrine dosage forms, the methods of the present invention, in certain embodiments, comprise the administration of buccal, lingual or sublingual epinephrine dosage forms comprising an amount of epinephrine having similar bioequivalency to about 0.01 mg/Kg of epinephrine administered by intra-muscular administration. In other embodiments, the buccal, lingual or sublingual dosage forms comprise an amount of epinephrine that is bioequivalent to about 0.10 mg to about 0.50 mg of epinephrine administered by intramuscular injection. In one embodiment, the buccal, lingual or sublingual dosage forms comprise an amount of epinephrine that is bioequivalent to about 0.10 mg of epinephrine administered by intra-muscular injection. In another embodiment, the buccal, lingual or sublingual dosage forms comprise an amount of epinephrine that is bioequivalent to about 0.15 mg of epinephrine administered by intramuscular injection. In still another embodiment, the buccal, lingual or sublingual dosage forms comprise an amount of epinephrine that is bioequivalent to about 0.30 mg of epinephrine administered by intra-muscular injection. In yet another embodiment, the buccal, lingual or sublingual dosage forms comprise an amount of epinephrine that is bioequivalent to about 0.45 mg of epinephrine administered by intra-muscular injection. In still yet another embodiment, the buccal, lingual or sublingual dosage forms comprise an amount of epinephrine that is bioequivalent to about 0.50 mg of epinephrine administered by intra-muscular injection. In yet other embodiments, the buccal, lingual or sublingual dosage forms comprise from about 1 mg to about 100 mg of epinephrine. In still other embodiments, the buccal, lingual or sublingual dosage form comprises from about 15 mg to about 60 mg of epinephrine.

In some embodiments, the buccal, lingual or sublingual doses administered can be approximately the same strength. In other embodiments, the buccal, lingual or sublingual epinephrine doses can decrease in strength (e.g., the second dose contains less epinephrine than the first, and the third dose (when present) contains less epinephrine than the first or second dose, etc.). In still other embodiments, the buccal, lingual or sublingual epinephrine doses can increase in strength (e.g., the second dose contains more epinephrine than the first, and the third dose (when present) contains more epinephrine than the first or second dose, etc.).

In certain embodiments of the present disclosure, the buccal, lingual or sublingual dosage forms can be self-administered by the patient. In other embodiments, the buccal, lingual or sublingual dosage forms can be administered to a patient by another person, such as a parent, a guardian, a care giver, or a health care professional. In certain embodiments, such healthcare professionals administer in an emergency setting, such as in the field, including ambulances or at a patient's home, etc.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.

Injectable Epinephrine Dosage Forms

The present invention provides methods of treating an allergic emergency, such as anaphylaxis, in a patient, comprising administering to the patient a series of buccal, lingual or sublingual dosage forms of epinephrine in combination with one or more injectable epinephrine dosage forms. The methods described herein can be practiced using any pharmaceutical solution comprising epinephrine that is appropriate for subcutaneous or intra-muscular injection. In some embodiments, aside from epinephrine, the epinephrine solution also contains at least one pharmaceutically inactive ingredient, such as sodium bisulfite as a preservative, a pH buffer, an agent for adjusting osmolality (such as to establish or maintain isotonicity with the tissue in which the solution is to be injected), or a mixture of two or more of the foregoing. Thus, as used herein, unless otherwise defined, the term “epinephrine solution” means an aqueous solution of epinephrine, which optionally comprises one or more additional ingredients other than epinephrine and water, such as preservative, buffer, and an agent for adjusting osmolality. As would be understood by one of skill in the art, different epinephrine solution concentrations can be used by adjusting the volume of epinephrine solution injected. Contemplated by the present invention is the administration of different epinephrine concentrations in different doses. Also contemplated by the present invention is the administration of different volumes of epinephrine solution in different doses.

As described throughout the specification and claims, the epinephrine solutions used in the methods provided herein can be administered from the same or different devices. Specifically, the first injectable dose can be administered from the same device as a subsequent injectable dose (e.g., optional second, optional third, etc.). Alternatively, the first injectable dose can be administered from a different device than a subsequent injectable dose (e.g., optional second, optional third, etc.). Thus, as a non-limiting example, certain embodiments can comprise the use of two single-use auto injector syringes, each comprising an injectable epinephrine dosage form.

In certain embodiments, each injectable dose of epinephrine solution can comprise from about 0.10 mg to about 0.60 mg of epinephrine. In still other embodiments, each injectable dose of epinephrine solution can comprise from about 0.20 mg to about 0.50 mg of epinephrine. In one embodiment, each injectable dose of epinephrine solution comprises about 0.10 mg of epinephrine. In another embodiment, each injectable dose of epinephrine solution comprises about 0.15 mg of epinephrine. In yet another embodiment, each injectable dose of epinephrine solution comprises about 0.30 mg of epinephrine. In still another embodiment, each injectable dose of epinephrine solution comprises about 0.40 mg of epinephrine. In still yet another embodiment, each injectable dose of epinephrine solution comprises about 0.45 mg of epinephrine. In yet another embodiment, each injectable dose of epinephrine solution comprises about 0.5 mg of epinephrine. By way of a non-limiting example, in certain embodiments, the injectable doses of the present invention can comprise 0.6 mg of epinephrine in 2 mLs of solution. In certain other embodiments, the injectable doses of the present invention can comprise 0.4 mg of epinephrine in 1.5 mLs of solution. In still other embodiments, the injectable doses of the present invention can comprise 0.5 mg of epinephrine in 0.5 mLs of solution. In yet still other embodiments, the injectable doses of the present invention can comprise 0.5 mg of epinephrine in 0.1 mLs of solution. However, as will be appreciated by those of skill in the art, the injectable doses of the present invention are not intended to be limited to any specific volumetric ranges.

In other embodiments, each injectable dose of epinephrine solution can comprise from about 0.25 mg/mL to about 5.0 mg/mL of epinephrine. In still other embodiments, each injectable dose of epinephrine solution can comprise from about 0.5 mg/mL to about 3.0 mg/mL of epinephrine. In yet other embodiments, each injectable dose of epinephrine solution can comprise about 0.5 mg/mL to about 1.5 mg/mL of epinephrine. In certain embodiments, each injectable dose of epinephrine solution comprises about 1.0 mg/mL of epinephrine.

In some embodiments, each of the injectable doses administered are about the same or substantially the same strength. In other embodiments, the injectable epinephrine doses decrease in strength (e.g., the second dose contains less epinephrine than the first, and the third dose (when present) contains less epinephrine than the second dose, etc.). In still other embodiments, the injectable epinephrine doses increase in strength (e.g., the second dose contains more epinephrine than the first, and the third dose (when present) contains more epinephrine than the second dose, etc.). In yet other embodiments, the injectable epinephrine doses increase in strength and then decrease in strength (e.g., the second dose contains more epinephrine than the first dose and the third dose, if needed, contains less epinephrine than the second dose and, in some cases, also less epinephrine than the first dose)—the alternative is also possible where the injectable epinephrine doses decrease in strength and then increase in strength (e.g., the second dose contains less epinephrine than the first dose and the third dose, if needed, contains more epinephrine than the second dose, and in some cases, also more epinephrine than the first dose).

In certain embodiments of the present invention, the injectable epinephrine doses can be self-administered by the patient. In other embodiments, the injectable epinephrine doses can be administered to a patient by another person, such as a parent, a guardian, a care giver, or a health care professional. In certain embodiments, such healthcare professionals administer in an emergency setting, such as in the field, including ambulances or at a patient's home, etc.

In some aspects of the present invention, the time interval between consecutive doses can be the amount of time it takes to see a therapeutic effect in the patient. In other embodiments, the time interval between consecutive doses ranges from about 3 minutes to about 10 minutes. In one embodiment, the time interval between consecutive doses is about 5 minutes.

Single Dosing of Epinephrine Solution with Manual or Automatic Devices

As described throughout the specification, the injectable doses described in the methods provided herein can each be single injector manual or automatic devices comprising epinephrine. In certain embodiments, the single injector is a manual device that is pre-assembled with epinephrine. In other embodiments, the single injector is a manual device, such as a syringe that is filled by a user from a vial comprising an epinephrine solution using syringe-filling techniques well known to those of skill in the art.

In certain other embodiments, the single injector is an automatic device that is pre-assembled with epinephrine. In still other embodiments, the single injector is an automatic device which is filled by a user from a vial comprising an epinephrine solution using syringe-filling techniques well known to those of skill in the art.

Multiple Dosing of Epinephrine Solution with Automatic-Automatic Devices

As described throughout the specification, the injectable doses described in the methods provided herein can each be automatically injected from the same device. Devices useful for these embodiments are those having the capability of injecting a plurality of medicament dosages. Examples of automatic-automatic injectors useful in the methods described herein can be found throughout the art and include, for example, those described in U.S. Pat. Nos. 3,572,336; 3,721,301; 3,882,863; 4,031,893; 4,226,235; 4,394,863; 4,723,937; 5,358,489; 5,540,664; and 5,665,071 and U.S. Published Application Nos: 20060173408; and 20060129122 (each of which is incorporated by reference herein in its entirety).

Additionally, devices having a single chamber for the epinephrine solution used in both the first and second doses, a single spring but capable of administering two different doses of medication, and a mechanism to prevent the administration of both the first and second dose at the same time are also useful in the present invention. In these embodiments, preferably the needle of the device retracts back when it is removed from the patient after automatic administration of the first dose. Alternatively, the needle of the device can be removed after automatic administration of the first dose. The tension left in the spring of such devices is sufficient to re-inject the same needle or a new needle into the patient upon administration of the second dose.

Multiple Dosing of Epinephrine Solution with Automatic-Manual Devices

As described throughout the specification, a device useful in the methods described herein is one that is capable of delivering the first dose of epinephrine solution automatically and delivering the second dose of epinephrine solution manually from the same device. Devices useful for these embodiments are those having the capability of injecting a plurality of medicament dosages. Examples of automatic-manual injectors useful in the methods described herein can be found throughout the art and include, for example, those described in U.S. Pat. Nos. 5,358,489; 5,540,664; 5,665,071; 5,695,472; and 5,833,669 and U.S. Published Application Nos: 20060173408; and 20060129122 (each of which is incorporated by reference herein in its entirety).

Multiple Dosing of Epinephrine Solution with Manual-Automatic Devices

As described throughout the specification, a device useful in the methods described herein is one that is capable of delivering a first dose of epinephrine solution manually and delivering a second dose of epinephrine solution automatically from the same device. Devices useful for these embodiments are those having the capability of injecting a plurality of medicament dosages. Examples of manual-automatic injectors useful in the methods described herein can be found throughout the art. Moreover, one of skill in the art would, with the teachings found throughout this application and those in the art, understand how to make and use a manual-automatic device useful in the present invention.

The following is a description of but one first dose manual injection, and a second dose automatic injection device useful in the present invention. The described uses are both possible using the same or similar procedures with a single fixed needle syringe or a double needle syringe as those devices are known and described in the art. To perform the first dose manual injection, the user (patient or someone other than the patient) manually inserts the forward needle into the flesh of the patient and depresses the plunger rod, preferably with the thumb. In certain embodiments, the first dose manual injection employs a stop collar to stop the plunger assembly of the syringe subassembly at a specific point to provide the desired amount of drug for injection. After the injection of the first manual dose, the stop collar is removed from the syringe subassembly to allow the plunger's further movement for a second dose automatic injection.

Multiple Dosing of Epinephrine Solution with Manual-Manual Devices

As described throughout the specification, the first and second doses described in the methods provided herein can each be manually injected from the same device. Devices useful for these embodiments are those having the capability of injecting a plurality of medicament dosages. Examples of manual-manual injectors useful in the methods described herein can be found throughout the art and include, for example, those described in U.S. Pat. Nos. 5,358,489; 5,540,664; 5,722,956; 5,232,459; and 5,665,071 (each of which are incorporated by reference herein in their entirety); PCT Application No. WO 88/07874 (which is incorporated by reference herein in its entirety); and US Published Application No. 2003/0004467 (which is incorporated by reference herein in its entirety).

Multiple Dosing of Epinephrine Solution

In embodiments where the administration of more than two doses is contemplated or is optional, devices useful in the invention are those capable of administering more than two doses, e.g., three doses. These devices include any combination of auto and manual devices, for example (but not limited to) auto-auto-auto, manual-manual-manual, or auto-auto-manual. In some embodiments, the different doses are administered from different devices (such as those described in the above sections entitled Multiple Dosing of Epinephrine Solutions with Automatic-Automatic Devices, Multiple Dosing of Epinephrine Solution with Automatic-Manual Devices, Multiple Dosing of Epinephrine Solution with Manual-Automatic Devices, and Multiple Dosing of Epinephrine Solution with Manual-Manual Devices). For example, where the method comprises administering three doses, two doses may be administered from the same device and one dose from a different device. Alternatively, all three doses may be administered from the same device, or all three doses may be administered from different devices. Some devices useful in the administration of two or more doses of epinephrine solution use multiple stop collars. For example, where three doses will be administered from the same device, that device may have two stop collars to allow for accurate administration of the three different doses, or even three stop collars, where the first stop collar is removed before a first dose of epinephrine solution is injected.

Epinephrine Dosing Regimens Comprising Buccal, Lingual or Sublingual Dosage Forms in Combination with Injectable Dosage Forms

In some embodiments, the present invention provides methods of treating an allergic emergency, such as anaphylaxis in a patient, comprising the steps of (a) administering to the patient at least one dose of a buccal, lingual or sublingual dosage form comprising epinephrine; and (b) administering to the patient at least one dose of an injectable dosage form comprising epinephrine. In certain embodiments, the methods comprise first administering to said patient at least one dose of a buccal, lingual or sublingual dosage form comprising epinephrine and subsequently administering to said patient at least one dose of an injectable dosage form comprising epinephrine. In certain other embodiments, the methods comprise first administering to said patient at least one dose of an injectable dosage form comprising epinephrine and subsequently administering to said patient at least one dose of a buccal, lingual or sublingual dosage form comprising epinephrine.

In other embodiments, the present invention provides methods for treating an allergic emergency in a patient comprising the steps of (a) administering to the patient one dose of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine. In another embodiment, the patient is administered a first and second dose of an injectable dosage form comprising epinephrine. In still another embodiment, the patient is administered a first, second, and third dose of an injectable dosage form comprising epinephrine.

In certain embodiments, the present invention provides methods for treating an allergic emergency in a patient comprising the steps of (a) administering to the patient two doses of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine. In another embodiment, the patient is administered a first and second dose of an injectable dosage form comprising epinephrine. In still another embodiment, the patient is administered a first, second, and third dose of an injectable dosage form comprising epinephrine.

In certain embodiments, the present invention provides methods for treating an allergic emergency in a patient comprising the steps of (a) administering to the patient three doses of a buccal, lingual or sublingual dosage form comprising epinephrine; and (b) administering to the patient a first dose of an injectable dosage form comprising epinephrine; (c) optionally, administering to the patient a second dose of an injectable dosage form comprising epinephrine; and (d) optionally, administering to the patient a third dose of an injectable dosage form comprising epinephrine. In one embodiment, the patient is administered a first dose of an injectable dosage form comprising epinephrine. In another embodiment, the patient is administered a first and second dose of an injectable dosage form comprising epinephrine. In still another embodiment, the patient is administered a first, second, and third dose of an injectable dosage form comprising epinephrine.

In other embodiments, the present invention provides methods for treating an allergic emergency in a patient comprising the steps of (a) administering to the patient one dose of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain other embodiments, the present invention provides methods for treating an allergic emergency in a patient comprising the steps of (a) administering to the patient two doses of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain other embodiments, the present invention provides methods for treating an allergic emergency in a patient comprising the steps of (a) administering to the patient three doses of an injectable dosage form comprising epinephrine; and (b) administering to the patient a first dose of a buccal, lingual or sublingual dosage form comprising epinephrine; (c) optionally, administering to the patient a second dose of a buccal, lingual or sublingual dosage form comprising epinephrine; and (d) optionally, administering to the patient a third dose of a buccal, lingual or sublingual dosage form comprising epinephrine. In one embodiment, the patient is administered a first dose of a buccal, lingual or sublingual dosage form comprising epinephrine. In another embodiment, the patient is administered a first and second dose of a buccal, lingual or sublingual dosage form comprising epinephrine. In still another embodiment, the patient is administered a first, second, and third dose of a buccal, lingual or sublingual dosage form comprising epinephrine.

In certain embodiments, the buccal, lingual or sublingual dosage forms can comprise an amount of epinephrine that is bioequivalent to about 0.01 mg/Kg of epinephrine administered by intra-muscular administration. In other embodiments, the buccal, lingual or sublingual dosage forms comprise an amount of epinephrine that is bioequivalent to about 0.10 mg to about 0.50 mg of epinephrine administered by intramuscular injection. In one embodiment, the buccal, lingual or sublingual dosage forms comprise an amount of epinephrine that is bioequivalent to about 0.10 mg of epinephrine administered by intra-muscular injection. In another embodiment, the buccal, lingual or sublingual dosage forms comprise an amount of epinephrine that is bioequivalent to about 0.15 mg of epinephrine administered by intra-muscular injection. In still another embodiment, the buccal, lingual or sublingual dosage forms comprise an amount of epinephrine that is bioequivalent to about 0.30 mg of epinephrine administered by intra-muscular injection. In yet another embodiment, the buccal, lingual or sublingual dosage forms comprise an amount of epinephrine that is bioequivalent to about 0.45 mg of epinephrine administered by intra-muscular injection. In still yet another embodiment, the buccal, lingual or sublingual dosage forms comprise an amount of epinephrine that is bioequivalent to about 0.50 mg of epinephrine administered by intra-muscular injection. In some embodiments, the buccal, lingual or sublingual dosage forms comprise from about 1 mg to about 100 mg of epinephrine. In other embodiments, the buccal, lingual or sublingual dosage forms comprise from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intra-muscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intra-muscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intra-muscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intra-muscular injection.

In other aspects of the present invention, the buccal, lingual or sublingual dosage forms comprising epinephrine can further comprise pharmaceutically acceptable excipients. In some embodiments, the buccal, lingual or sublingual dosage forms further comprise absorption enhancers. In certain embodiments, the absorption enhancer can be a transmucosal absorption enhancer. Transmucosal absorption enhancers are known in the art and include, but are not limited to, chelators (e.g., EDTA, EGTA), non-ionic surfactants (e.g., 23-lauryl ether, laureth-9, polysorbates (including polysorbate 80), sucrose esters, or dodecylmaltoside), cationic surfactants (e.g., benzalkonium chloride or cetylmethylammonium bromide), anionic surfactants (e.g., sodium dodecyl glycocholate or sodium lauryl sulfate), bile salts and other steroidal detergents (e.g., cholate, deoxycholate, taurocholate, sodium glycocholate, sodium taurocholate, saponins, sodium taurodihydrofusidate or sodium glycodihydrofusidate), fatty acids (e.g., oleic acid, lauric acid capric acid, heptanoic acid, stearic acid, sucrose laurate, isopropyl myristate, sodium myristate or caprylic acid), and non-surfactants (e.g., aprotinin, dextran sulfate, sulfoxides, salicylates, Intravail® or 1-dodecylazacycloheptane-2-one (Azone)), phospholipids (e.g., phosphatidylcholines, lysophosphatidylcholine, or monoooleoyl phosphaltidyl ethanomamine), cyclodextrins, and various alkyl glycosides. See, e.g., Shojaei, 1998, J Pharm Pharmaceut Sci 1:15-30; and Mitra et al., 2002, Encyclopedia of Pharmaceutical Technology pp. 2081-2095. In certain embodiments, the transmucosal absorption enhancer can be Intravail® (Aegis Therapeutics, LLC, San Diego, Calif.). In other embodiments, the transmucosal absorption enhancer can be benzalkonium chloride.

In certain embodiments, each injectable dose of epinephrine solution can comprise from about 0.10 mg to about 0.60 mg of epinephrine. In still other embodiments, each injectable dose of epinephrine solution can comprise from about 0.20 mg to about 0.50 mg of epinephrine. In one embodiment, each injectable dose of epinephrine solution comprises about 0.10 mg of epinephrine. In another embodiment, each injectable dose of epinephrine solution comprises about 0.15 mg of epinephrine. In yet another embodiment, each injectable dose of epinephrine solution comprises about 0.30 mg of epinephrine. In still another embodiment, each injectable dose of epinephrine solution comprises about 0.40 mg of epinephrine. In still yet another embodiment, each injectable dose of epinephrine solution comprises about 0.45 mg of epinephrine. In yet another embodiment, each injectable dose of epinephrine solution comprises about 0.5 mg of epinephrine. By way of certain non-limiting examples, the injectable doses of the present invention can comprise 0.6 mg of epinephrine in 2 mLs of solution. In certain other embodiments, the injectable doses of the present invention can comprise 0.4 mg of epinephrine in 1.5 mLs of solution. In still other embodiments, the injectable doses of the present invention can comprise 0.5 mg of epinephrine in 0.5 mLs of solution. In yet still other embodiments, the injectable doses of the present invention can comprise 0.5 mg of epinephrine in 0.1 mLs of solution. However, as will be appreciated by those of skill in the art, the injectable doses of the present invention are not intended to be limited to any specific volumetric ranges.

In some embodiments, each injectable dose of epinephrine solution can comprise from about 0.25 mg/mL to about 5.0 mg/mL of epinephrine. In still other embodiments, each injectable dose of epinephrine solution can comprise from about 0.5 mg/mL to about 3.0 mg/mL of epinephrine. In yet other embodiments, each injectable dose of epinephrine solution can comprise about 0.5 mg/mL to about 1.5 mg/mL of epinephrine. In one embodiment, each injectable dose of epinephrine solution comprises about 1.0 mg/mL of epinephrine.

In other embodiments, the injectable epinephrine dosage form can comprise an epinephrine solution further comprising at least one pharmaceutically inactive ingredient, such as sodium bisulfite as a preservative, a pH buffer, an agent for adjusting osmolality (such as to establish or maintain isotonicity with the tissue in which the solution is to be injected), or a mixture of two or more of the foregoing.

In other aspects of the present invention, the methods provided herein can comprise injectable dosage forms wherein the epinephrine solution can be delivered by either an automatic injection or a manual injection. In certain embodiments, manual injections can be from a pre-filled manual injector, such as a syringe, or from an empty or partially-filled syringe that is filled by a user from a vial comprising an epinephrine solution using syringe-filling techniques well known to those of skill in the art. For example, the first injectable dose can be delivered by either an automatic injection or a manual injection, the second injectable dose can be delivered by either an automatic injection or a manual injection (if required), and the third injectable dose can be delivered by either an automatic injection or a manual injection (if required). In some embodiments, the first injectable dose and second injectable dose can be delivered by automatic injection from the same device and the third injectable dose can be delivered either automatically or manually from a different device. In other embodiments, the first injectable dose and the second injectable dose can be delivered by manual injection from the same device and the third injectable dose can be delivered either automatically or manually from a different device. In still other embodiments, the first injectable dose can be administered by automatic injection and the second injectable dose can be delivered by manual injection and the third injectable dose can be delivered either automatically or manually from a different device, or the first injectable dose can be administered by manual injection and the second injectable dose by automatic injection and the third injectable dose can be delivered either automatically or manually from a different device.

In some embodiments, the second or more dose of a buccal, lingual or sublingual dosage form comprising epinephrine comprises an amount of epinephrine greater than the first dose of a buccal, lingual or sublingual dosage form comprising epinephrine. In other embodiments, the second or more dose of a buccal, lingual or sublingual dosage form comprising epinephrine comprises an amount of epinephrine less than the first dose of a buccal, lingual or sublingual dosage form comprising epinephrine. In still other embodiments, the second or more dose of a buccal, lingual or sublingual dosage form comprising epinephrine comprises an amount of epinephrine that is from about 25% to about 200% of the amount of epinephrine in the first dose of a buccal, lingual or sublingual dosage form comprising epinephrine.

In some embodiments, the second or more dose of a buccal, lingual or sublingual dosage form comprising epinephrine comprises an amount of epinephrine greater than the preceding dose of a buccal, lingual or sublingual dosage form comprising epinephrine. In other embodiments, the second or more dose of a buccal, lingual or sublingual dosage form comprising epinephrine comprises an amount of epinephrine less than the preceding dose of a buccal, lingual or sublingual dosage form comprising epinephrine. In still other embodiments, the second or more dose of a buccal, lingual or sublingual dosage form comprising epinephrine comprises an amount of epinephrine that is from about 25% to about 200% of the amount of epinephrine in the preceding dose of a buccal, lingual or sublingual dosage form comprising epinephrine.

In still other embodiments, the second or more dose of an injectable dosage form comprising epinephrine comprises an amount of epinephrine greater than the first dose of an injectable dosage form comprising epinephrine. In other embodiments, the second or more dose of an injectable dosage form comprising epinephrine comprises an amount of epinephrine less than the first dose of an injectable dosage form comprising epinephrine. In still other embodiments, the second or more dose of an injectable dosage form comprising epinephrine comprises an amount of epinephrine that is from about 25% to about 200% of the amount of epinephrine in the first dose of an injectable dosage form comprising epinephrine.

In yet still other embodiments, the second or more dose of an injectable dosage form comprising epinephrine comprises an amount of epinephrine greater than the preceding dose of an injectable dosage form comprising epinephrine. In other embodiments, the second or more dose of an injectable dosage form comprising epinephrine comprises an amount of epinephrine less than the preceding dose of an injectable dosage form comprising epinephrine. In still other embodiments, the second or more dose of an injectable dosage form comprising epinephrine comprises an amount of epinephrine that is from about 25% to about 200% of the amount of epinephrine in the preceding dose of an injectable dosage form comprising epinephrine.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.

Rectal Dosing Regimens of Epinephrine for the Treatment of Anaphylaxis

In some embodiments, the present invention provides a method of treating an allergic emergency, such as anaphylaxis in a patient, comprising the steps of (a) administering to the patient at least one dose of a rectal dosage form comprising epinephrine; and (b) administering to the patient at least one or more dose of an injectable dosage form comprising epinephrine. In certain embodiments, the methods comprise first administering to said patient at least one dose of a rectal dosage form comprising epinephrine and subsequently administering to said patient at least one dose of an injectable dosage form comprising epinephrine. In certain other embodiments, the methods comprise first administering to said patient at least one dose of an injectable dosage form comprising epinephrine and subsequently administering to said patient at least one dose of a rectal dosage form comprising epinephrine.

The rectal dosage forms useful for the methods described herein include, but are not limited to, suppositories, rectal capsules, gels, creams, and ointments. In certain embodiments, the rectal dosage forms further comprise a pharmaceutically acceptable excipient. In one embodiment, the rectal dosage form is a suppository comprising epinephrine and a pharmaceutically acceptable excipient.

In certain embodiments, the rectal epinephrine dosage forms comprising epinephrine can be bioequivalent to about 0.01 mg/Kg of epinephrine administered by intra-muscular administration. In other embodiments, the rectal dosage forms comprise an amount of epinephrine that is bioequivalent to about 0.10 mg to about 0.50 mg of epinephrine administered by intra-muscular injection. In one embodiment, the rectal dosage forms comprise an amount of epinephrine that is bioequivalent to about 0.10 mg of epinephrine administered by intra-muscular injection. In another embodiment, the rectal dosage forms comprise an amount of epinephrine that is bioequivalent to about 0.15 mg of epinephrine administered by intra-muscular injection. In still another embodiment, the rectal dosage forms comprise an amount of epinephrine that is bioequivalent to about 0.30 mg of epinephrine administered by intra-muscular injection. In yet another embodiment, the rectal dosage forms comprise an amount of epinephrine that is bioequivalent to about 0.45 mg of epinephrine administered by intra-muscular injection. In still yet another embodiment, the rectal dosage forms comprise an amount of epinephrine that is bioequivalent to about 0.50 mg of epinephrine administered by intra-muscular injection. In yet other embodiments, the rectal dosage forms comprise from about 1 mg to about 100 mg of epinephrine. In still other embodiments, the rectal dosage form comprises from about 15 mg to about 60 mg of epinephrine.

In other embodiments of the present invention, the subsequent administration of a second or greater rectal dosage form is bioequivalent to the subsequent administration of a second or greater injectable dosage form. In certain embodiments, the subsequent administration of a second or greater rectal dosage form is bioequivalent to the subsequent administration of a second or greater injectable dosage form comprising about 0.01 mg/Kg of epinephrine administered by intra-muscular administration. In other embodiments, the subsequent administration of a second or greater rectal dosage form is bioequivalent to the subsequent administration of a second or greater injectable dosage form comprising about 0.10 mg to about 0.50 mg of epinephrine administered by intra-muscular injection. In still other embodiments, the subsequent administration of a second or greater rectal dosage form is bioequivalent to the subsequent administration of a second or greater injectable dosage form comprising about 0.15 mg epinephrine administered by intra-muscular injection. In yet other embodiments, the subsequent administration of a second or greater rectal dosage form is bioequivalent to the subsequent administration of a second or greater injectable dosage form comprising about 0.30 epinephrine administered by intra-muscular injection. In yet still other embodiments, the subsequent administration of a second or greater rectal dosage form is bioequivalent to the subsequent administration of a second or greater injectable dosage form comprising about 0.50 mg of epinephrine administered by intra-muscular injection.

In other embodiments, the rectal dosage forms comprise an amount of epinephrine that is bioequivalent to about 0.1 mg to about 0.5 mg of epinephrine administered by intramuscular injection. In one embodiment, the rectal dosage forms comprise an amount of epinephrine that is bioequivalent to about 0.15 mg of epinephrine administered by intramuscular injection. In another embodiment, the rectal dosage forms comprise an amount of epinephrine that is bioequivalent to about 0.3 mg of epinephrine administered by intra-muscular injection or administration. In still another embodiment, the rectal dosage forms comprise about 1 mg to about 100 mg of epinephrine. In yet another embodiment, the rectal dosage forms comprise about 15 mg to about 60 mg of epinephrine.

In certain embodiments, each injectable dose of epinephrine solution can comprise from about 0.10 mg to about 0.60 mg of epinephrine. In still other embodiments, each injectable dose of epinephrine solution can comprise from about 0.20 mg to about 0.50 mg of epinephrine. In one embodiment, each injectable dose of epinephrine solution comprises about 0.10 mg of epinephrine. In another embodiment, each injectable dose of epinephrine solution comprises about 0.15 mg of epinephrine. In yet another embodiment, each injectable dose of epinephrine solution comprises about 0.30 mg of epinephrine. In still another embodiment, each injectable dose of epinephrine solution comprises about 0.40 mg of epinephrine. In still yet another embodiment, each injectable dose of epinephrine solution comprises about 0.45 mg of epinephrine. In yet another embodiment, each injectable dose of epinephrine solution comprises about 0.5 mg of epinephrine. By way of certain non-limiting examples, the injectable doses of the present invention can comprise 0.6 mg of epinephrine in 2 mLs of solution. In other embodiments, the injectable doses of the present invention can comprise 0.4 mg of epinephrine in 1.5 mLs of solution. In still other embodiments, the injectable doses of the present invention can comprise 0.5 mg of epinephrine in 0.5 mLs of solution. In yet still other embodiments, the injectable doses of the present invention can comprise 0.5 mg of epinephrine in 0.1 mLs of solution. However, as will be appreciated by those of skill in the art, the injectable doses of the present invention are not intended to be limited to any specific volumetric ranges.

In some embodiments, each injectable dose of epinephrine solution can comprise from about 0.25 mg/mL to about 5.0 mg/mL of epinephrine. In still other embodiments, each injectable dose of epinephrine solution can comprise from about 0.5 mg/mL to about 3.0 mg/mL of epinephrine. In yet other embodiments, each injectable dose of epinephrine solution can comprise about 0.5 mg/mL to about 1.5 mg/mL of epinephrine. In one embodiment, each injectable dose of epinephrine solution comprises about 1.0 mg/mL of epinephrine.

In other aspects of the present invention, the methods provided herein can comprise injectable dosage forms wherein the epinephrine solution can be delivered by either an automatic injection or a manual injection. For example, the first injectable dose can be delivered by either an automatic injection or a manual injection, the second injectable dose can be delivered by either an automatic injection or a manual injection (if required), and the third injectable dose can be delivered by either an automatic injection or a manual injection (if required). In some embodiments, the first injectable dose and second injectable dose can be delivered by automatic injection from the same device and the third injectable dose can be delivered either automatically or manually from a different device. In other embodiments, the first injectable dose and the second injectable dose can be delivered by manual injection from the same device and the third injectable dose can be delivered either automatically or manually from a different device. In still other embodiments, the first injectable dose can be administered by automatic injection and the second injectable dose can be delivered by manual injection and the third injectable dose can be delivered either automatically or manually from a different device, or the first injectable dose can be administered by manual injection and the second injectable dose by automatic injection and the third injectable dose can be delivered either automatically or manually from a different device.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.

Kits and Packaging System Comprising Doses of Buccal, Lingual or Sublingual Epinephrine with Injectable Doses of Epinephrine

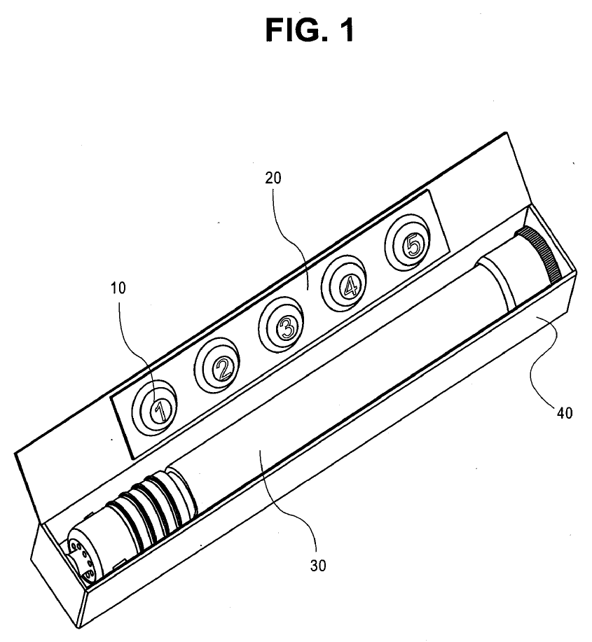

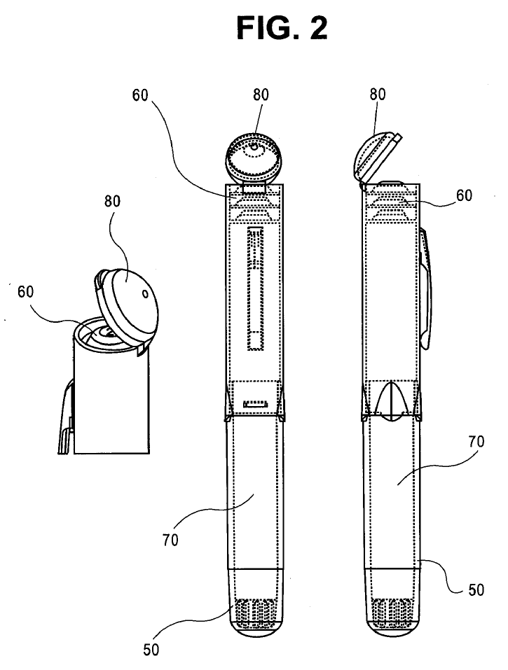

The present invention is further directed to a kit or packaging system for the administration of multiple doses of epinephrine comprising at least one dose of a buccal, lingual or sublingual dosage form comprising epinephrine and at least one dose of an injectable dosage form comprising epinephrine to a patient in need thereof, such as a patient experiencing anaphylaxis, an anaphylactoid reaction or a set of symptoms resembling anaphylaxis or anaphylactoid reaction of unknown etiology but suspected of being an allergic emergency. In certain embodiments, the present invention is directed to a kit or packaging system for use in the treatment of an allergic emergency in a patient, comprising (a) at least one dose of a buccal, lingual or sublingual dosage form comprising epinephrine; and (b) at least one dose of an injectable dosage form comprising epinephrine. A packaging system according to one embodiment of the invention is illustrated in FIG. 1, wherein the packaging system comprises five (5) dosage forms and two injectable dosage forms within an automatic-manual injector device. A second embodiment is provided in FIG. 2, wherein the packaging system comprises three (3) dosage forms and two injectable dosage forms within an automatic-manual injector device. As shown in FIG. 2, the dosage forms can be housed in a flip-top. In other embodiments, the dosage forms can be housed in a screw-top. In yet another embodiment, the dosage forms can be housed in a removable sliding top wherein the top is held in place by the friction fit of the upper and lower portions of the carrying case.

In some embodiments, the kit or packaging system further comprises written instructions for administering the at least one dose of a buccal, lingual or sublingual dosage form comprising epinephrine and the at least one dose of injectable dosage form comprising epinephrine.

In certain embodiments, the written instructions provide that (a) the at least one dose of a buccal, lingual or sublingual dosage form comprising epinephrine is first administered to said patient to provide a therapeutic effect in response to said allergic emergency; and optionally (b) subsequent to the first administration of the at least one dose of a buccal, lingual or sublingual dosage form comprising epinephrine, the at least one dose of an injectable dosage form comprising epinephrine is administered to said patient.

In certain other embodiments, the written instructions provide that (a) the at least one dose of an injectable dosage form comprising epinephrine is first administered to said patient to provide a therapeutic effect in response to said allergic emergency; and optionally (b) subsequent to the first administration of the at least one dose of an injectable dosage form comprising epinephrine, the at least one dose of a buccal, lingual or sublingual dosage form comprising epinephrine is administered to said patient.

In some embodiments, the at least one dose of a buccal, lingual or sublingual dosage form comprising epinephrine is contained within a protective liner. In one embodiment, the protective liner prevents damage due to moisture, light, or oxygen. In another embodiment, the protective liner is a polymer-lined foil. In another embodiment, the protective packaging comprises a blister package. In certain embodiments, the protective packaging comprises a blister package wherein each individual buccal, lingual, or sublingual dosage form is contained within an individual blister.

In yet other embodiments, the doses are identified in the kit or packaging system. In another embodiment, the doses are identified by numerical markings or by location within the kit or packaging system. In yet still another embodiment, the identification of the doses indicates the order in which the doses are administered to the patient. In other embodiments, the kit or packaging system further comprises a carrying case. In certain embodiments, the buccal, lingual or sublingual doses are identified by numerical marking that are embossed on the packaging system. In certain other embodiments, the buccal, lingual or sublingual doses are identified by numerical markings that are etched on the dosage form.

Alternately, in the above-described kits, the buccal, lingual, or sublingual dosage forms may instead be a rectal dosage form comprising epinephrine as described above.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following ingredients, processes and procedures for practicing the methods described herein correspond to that described above. The procedures below describe specific embodiments of methods of administering the buccal, lingual or sublingual epinephrine dosage forms and the injectable epinephrine dosage forms as described herein. Any methods or materials not particularly described in the following examples are within the scope of the invention and will be apparent to those skilled in the art with reference to the disclosure herein.

### Example 1

Co-Administration of Buccal and Injectable Dosage Forms Comprising Epinephrine for the Treatment of Anaphylaxis

A patient experiencing an allergic emergency initiates treatment at the onset of shortness of breath by self administering 40 mg of epinephrine free base in a buccal dosage form. After approximately 5 minutes pass without amelioration of the symptoms of anaphylaxis, the patient self administers a second 40 mg of epinephrine free base in a buccal dosage form. After another approximately 5 minutes pass without amelioration of the symptoms of anaphylaxis, the patient auto-injects a first dose 0.3 mg epinephrine from an injectable dosage form. Within about five minutes after the administration of the first injectable dose of epinephrine, the patient's symptoms of anaphylaxis are relieved.

### Example 2

Co-Administration of Buccal and Injectable Dosage Forms Comprising Epinephrine for the Treatment of Anaphylaxis

A patient experiencing an allergic emergency initiates treatment at the onset of shortness of breath by self administering 40 mg of epinephrine free base in a buccal dosage form. After approximately 5 minutes pass without amelioration of the symptoms of anaphylaxis, the patient self administers a second buccal dosage form comprising 60 mg of epinephrine free base. After another approximately 5 minutes pass without amelioration of the symptoms of anaphylaxis, the patient auto-injects a first dose 0.3 mg epinephrine from an injectable dosage form. After another approximately 5 minutes pass without amelioration of the symptoms of anaphylaxis, the patient auto-injects a second dose 0.3 mg epinephrine from an injectable dosage form. Within about five minutes after the administration of the second injectable dose of epinephrine, the patient's symptoms of anaphylaxis are relieved.

### Example 3

Co-Administration of Lingual and Injectable Dosage Forms Comprising Epinephrine for the Treatment of Anaphylaxis

A patient experiencing an allergic emergency initiates treatment at the onset of shortness of breath by self administering 40 mg of epinephrine free base in a lingual dosage form. After approximately 5 minutes pass without amelioration of the symptoms of anaphylaxis, the patient self administers a second lingual dosage form comprising 60 mg of epinephrine free base. After another approximately 5 minutes pass without amelioration of the symptoms of anaphylaxis, the patient auto-injects a first dose 0.3 mg epinephrine from an injectable dosage form. After another approximately 5 minutes pass without amelioration of the symptoms of anaphylaxis, the patient auto-injects a second dose 0.3 mg epinephrine from an injectable dosage form. Within about five minutes after the administration of the second injectable dose of epinephrine, the patient's symptoms of anaphylaxis are relieved.

### Example 4

A Kit Comprising Sublingual and Injectable Dosage Forms of Epinephrine for the Treatment of Anaphylaxis

A kit is provided which contains two doses of a sublingual dosage form each containing 40 mg of epinephrine and one dose of an automatic injectable dosage form comprising 0.3 mg epinephrine.

### Example 5

A Kit Comprising Sublingual and Injectable Dosage Forms of Epinephrine for the Treatment of Anaphylaxis

A kit is provided which contains two doses of a sublingual dosage form each containing 40 mg of epinephrine and one dose of an automatic injectable dosage form comprising 0.3 mg epinephrine. The sublingual dosage forms of the kit are packaged in a foil blister pack with numerical markings identifying the order and location of each dose. The automatic injectable dosage form is sealed in a foil-lined plastic pouch.

### Example 6

A Kit Comprising Sublingual and Injectable Dosage Forms of Epinephrine for the Treatment of Anaphylaxis

A kit is provided which contains two doses of a sublingual dosage form each containing 40 mg of epinephrine and one dose of an automatic injectable dosage form comprising 0.3 mg epinephrine. The sublingual dosage forms of the kit are packaged in a foil blister pack with numerical markings identifying the order and location of each dose. The automatic injectable dosage form is sealed in a foil-lined plastic pouch.

Specifically, the foil blister pack containing the two sublingual dosage forms is embossed with the numbers 1 and 2, respectively. The numerical markings provide easy identification of each dosage form by the patient. The automatic injectable dose is marked as dose 3 on both the injector's packaging and the injector's labeling.

### Example 7

A Kit Comprising Sublingual and Injectable Dosage Forms of Epinephrine for the Treatment of Anaphylaxis

A kit is provided which contains two doses of a sublingual dosage form each containing 40 mg of epinephrine and one dose of an automatic injectable dosage form comprising 0.3 mg epinephrine. The sublingual dosage forms of the kit are packaged in a foil blister pack with numerical markings identifying the order and location of each dose. The automatic injectable dosage form is sealed in a foil-lined plastic pouch.

Specifically, the foil blister pack containing the two sublingual dosage forms is embossed with the numbers 1 and 2, respectively. The numerical markings provide easy identification of each dosage form by the patient. The automatic injectable dose is marked as dose 3 on both the injector's packaging and the injector's labeling.

The kit further contains written instructions to aid the patient in administering the dosage forms of epinephrine contained therein in the correct order and at the correct time.

The instructions provide as follows: (a) the first sublingual dosage form, labeled as 1, is to be administered under the tongue of the patient and maintained there until fully dissolved as soon as the patient begins experiencing symptoms of anaphylaxis; (b) if the symptoms of anaphylaxis do not improve or terminate within approximately five minutes, the second sublingual dosage form, labeled as 2, is to be administered under the tongue of the patient and maintained there until fully dissolved; and (c) if the symptoms of anaphylaxis do not improve or terminate within approximately five minutes after administration of the second dose, the first automatic injectable dosage form, labeled as 3, is to be auto-injected.

The written instructions further provide standard information including the proper storage conditions for the dosage forms, how to properly dispose of the unused dosage forms, contra-indications related to dosage forms comprising epinephrine, etc.

A carrying case is also included in the kit which provides easy storage for the sublingual and injectable dosage forms and also provides additional protection from moisture, light and oxygen.

### Example 8

A Kit Comprising Sublingual and Injectable Dosage Forms of Epinephrine for the Treatment of Anaphylaxis

A kit is provided which contains two doses of a sublingual dosage form and three doses of a manual injectable dosage form. The first sublingual dose contains 40 mg of epinephrine. The second sublingual dose contains 60 mg of epinephrine. The first manual injectable dose contains about 0.3 mg of epinephrine. The second manual injectable dose contains about 0.3 mg of epinephrine. The third manual injectable dose contains about 0.3 mg of epinephrine.

### Example 9

A Kit Comprising Sublingual and Injectable Dosage Forms of Epinephrine for the Treatment of Anaphylaxis

A kit is provided which contains two doses of a sublingual dosage form and three doses of a manual injectable dosage form. The first sublingual dose contains 40 mg of epinephrine. The second sublingual dose contains 60 mg of epinephrine. The first manual injectable dose contains about 0.3 mg of epinephrine. The second manual injectable dose contains about 0.3 mg of epinephrine. The third manual injectable dose contains about 0.3 mg of epinephrine.

The sublingual dosage forms are packaged in a foil blister pack with numerical markings identifying the order and location of each dose. The manual injectable dosage forms are each sealed in a foil-lined plastic pouch.

Specifically, the foil blister pack containing the two sublingual dosage forms is embossed with the numbers 1 and 2, respectively. The numerical markings provide easy identification of each dosage form by the patient. The manual injectable doses are marked as dose 3, 4, and 5, respectively, on both injector's packaging and the injector's labeling.

### Example 10

A Kit Comprising Sublingual and Injectable Dosage Forms of Epinephrine for the Treatment of Anaphylaxis

A kit is provided which contains two doses of a sublingual dosage form and three doses of a manual injectable dosage form. The first sublingual dose contains 40 mg of epinephrine. The second sublingual dose contains 60 mg of epinephrine. The first manual injectable dose contains about 0.3 mg of epinephrine. The second manual injectable dose contains about 0.3 mg of epinephrine. The third manual injectable dose contains about 0.3 mg of epinephrine.

The sublingual dosage forms are packaged in a foil blister pack with numerical markings identifying the order and location of each dose. The manual injectable dosage forms are each sealed in a foil-lined plastic pouch.

Specifically, the foil blister pack containing the two sublingual dosage forms is embossed with the numbers 1 and 2, respectively. The numerical markings provide easy identification of each dosage form by the patient. The manual injectable doses are marked as dose 3, 4, and 5, respectively, on both injector's packaging and the injector's labeling.

The kit further contains written instructions to aid the patient in administering the dosage forms of epinephrine contained therein in the correct order and at the correct time.

The instructions provide as follows: (a) the first sublingual dosage form, labeled as 1, is to be administered under the tongue of the patient and maintained there until fully dissolved as soon as the patient begins experiencing symptoms of anaphylaxis; (b) if the symptoms of anaphylaxis do not improve or terminate within approximately five minutes, the second sublingual dosage form, labeled as 2, is to be administered under the tongue of the patient and maintained there until fully dissolved; (c) if the symptoms of anaphylaxis do not improve or terminate within approximately five minutes after administration of the second dose, the first injectable dosage form, labeled as 3, is to be manually injected; (d) if the symptoms of anaphylaxis do not improve or terminate within approximately five minutes after administration of the third dose, the second injectable dosage form, labeled as 4, is to be manually injected; and (e) if the symptoms of anaphylaxis do not improve or terminate within approximately five minutes after administration of the fourth dose, the third injectable dosage form, labeled as 5, is to be manually injected.

The written instructions further provide standard information including the proper storage conditions for the dosage forms, how to properly dispose of the unused dosage forms, contra-indications related to dosage forms comprising epinephrine, etc.

A carrying case is also included in the kit which provides easy storage for the sublingual and injectable dosage forms and also provides additional protection from moisture, light and oxygen.

What is claimed is:

1. A method for treating an allergic emergency in a patient, comprising
a. administering to said patient at least one dose of a buccal, lingual or sublingual dosage form comprising epinephrine; and
b. administering to said patient at least one dose of an injectable dosage form comprising epinephrine.

2. The method of claim 1, wherein said method comprises first administering to said patient said at least one dose of a buccal, lingual or sublingual dosage form comprising epinephrine and subsequently administering to said patient said at least one dose of an injectable dosage form comprising epinephrine.

3. The method of claim 1, wherein said method comprises first administering to said patient said at least one dose of an injectable dosage form comprising epinephrine and subsequently administering to said patient said at least one dose of a buccal, lingual or sublingual dosage form comprising epinephrine.

4. The method of claim 2, wherein said method comprises:
a. administering to said patient one (1) dose of a buccal, lingual or sublingual dosage form comprising epinephrine;
b. administering to said patient a first dose of an injectable dosage form comprising epinephrine;
c. optionally, administering to said patient a second dose of an injectable dosage form comprising epinephrine; and
d. optionally, administering to said patient a third dose of an injectable dosage form comprising epinephrine.

5. The method of claim 4, wherein said patient is administered a first dose of an injectable dosage form comprising epinephrine.

6. The method of claim 4, wherein said patient is administered a first and second dose of an injectable dosage form comprising epinephrine.

7. The method of claim 4, wherein said patient is administered a first, second and third dose of an injectable dosage form comprising epinephrine.

8. The method of claim 2, wherein said method comprises:
a. administering to said patient two (2) doses of a buccal, lingual or sublingual dosage form comprising epinephrine;
b. administering to said patient a first dose of an injectable dosage form comprising epinephrine;
c. optionally, administering to said patient a second dose of an injectable dosage form comprising epinephrine; and
d. optionally, administering to said patient a third dose of an injectable dosage form comprising epinephrine.

9. The method of claim 8, wherein said patient is administered a first dose of an injectable dosage form comprising epinephrine.

10. The method of claim 8, wherein said patient is administered a first and second dose of an injectable dosage form comprising epinephrine.

11. The method of claim 8, wherein said patient is administered a first, second and third dose of an injectable dosage form comprising epinephrine.

12. The method of claim 3, wherein said method comprises:
a. administering to said patient one (1) dose of an injectable dosage form comprising epinephrine;
b. administering to said patient a first dose of a buccal, lingual or sublingual dosage form comprising epinephrine;
c. optionally, administering to said patient a second dose of a buccal, lingual or sublingual dosage form comprising epinephrine; and
d. optionally, administering to said patient a third dose of a buccal, lingual or sublingual dosage form comprising epinephrine.

13. The method of claim 12, wherein said patient is administered a first dose of a buccal, lingual or sublingual dosage form comprising epinephrine.

14. The method of claim 12, wherein said patient is administered a first and second dose of a buccal, lingual or sublingual dosage form comprising epinephrine.

15. The method of claim 12, wherein said patient is administered a first, second and third dose of a buccal, lingual or sublingual dosage form comprising epinephrine.

16. The method of claim 3, wherein said method comprises:
a. administering to said patient two (2) doses of an injectable dosage form comprising epinephrine;
b. administering to said patient a first dose of a buccal, lingual or sublingual dosage form comprising epinephrine;
c. optionally, administering to said patient a second dose of a buccal, lingual or sublingual dosage form comprising epinephrine; and
d. optionally, administering to said patient a third dose of a buccal, lingual or sublingual dosage form comprising epinephrine.

17. The method of claim 16, wherein said patient is administered a first dose of a buccal, lingual or sublingual dosage form comprising epinephrine.

18. The method of claim 16, wherein said patient is administered a first and second dose of a buccal, lingual or sublingual dosage form comprising epinephrine.

19. The method of claim 16, wherein said patient is administered a first, second and third dose of a buccal, lingual or sublingual dosage form comprising epinephrine.

20. The method of claim 1, wherein each said buccal, lingual or sublingual dosage form comprises an amount of epinephrine bioequivalent to about 0.01 mg/Kg of epinephrine administered by intramuscular administration.

21. The method of claim 1, wherein each said buccal, lingual or sublingual dosage form comprises an amount of epinephrine bioequivalent to about 0.15 mg of epinephrine administered by intramuscular administration

22. The method of claim 1, wherein each said buccal, lingual or sublingual dosage form comprises an amount of epinephrine bioequivalent to about 0.3 mg of epinephrine administered by intramuscular administration

23. The method of claim 1, wherein each said buccal, lingual or sublingual dosage form comprises from about 1 mg to about 100 mg of epinephrine.

24. The method of claim 23, wherein each said buccal, lingual or sublingual dosage form comprises from about 15 mg to about 60 mg of epinephrine.

25. The method of claim 1, wherein each said buccal, lingual or sublingual dosage form is a tablet.

26. The method of claim 25, wherein the dosage form is a sublingual tablet.

27. The method of claim 1, wherein each said buccal, lingual or sublingual dosage form further comprises a pharmaceutically acceptable excipient.

28. The method of claim 1, wherein each said injectable dosage form comprises from about 0.25 mg/mL to about 5.0 mg/mL of epinephrine.

29. The method of claim 28, wherein each said injectable dosage form comprises from about 0.5 mg/mL to about 3.0 mg/mL of epinephrine.

30. The method of claim 28, wherein each said injectable dosage form comprises from about 0.5 mg/mL to about 1.5 mg/mL of epinephrine.

31. The method of claim 28, wherein each said injectable dosage form comprises about 1.0 mg/mL of epinephrine.

32. The method of claim 1, wherein each said injectable dosage form comprises from about 0.1 mg to about 0.6 mg of epinephrine.

33. The method of claim 32, wherein each said injectable dosage form comprises about 0.15 mg of epinephrine.

34. The method of claim 32, wherein each said injectable dosage form comprises about 0.3 mg of epinephrine.

35. The method of claim 32, wherein each said injectable dosage form comprises about 0.5 mg of epinephrine.

36. The method of claim 1, wherein each said injectable dosage form further comprises at least one pharmaceutically inactive ingredient.

37. The method of claim 1, wherein said at least one dose of an injectable dosage form is automatically injected.

38. The method of claim 1, wherein said at least one dose of an injectable dosage form is manually injected.

39. The method of claim 1, wherein said administering of said dosage forms comprising epinephrine is carried out by said patient.

40. The method of claim 1, wherein the second or more dose of said buccal, lingual or sublingual dosage form comprising epinephrine comprises an amount of epinephrine greater than the first dose of said buccal, lingual or sublingual dosage form comprising epinephrine.

41. The method of claim 1, wherein the second or more dose of said buccal, lingual or sublingual dosage form comprising epinephrine comprises an amount of epinephrine less than the first dose of said buccal, lingual or sublingual dosage form comprising epinephrine.

42. The method of claim 1, wherein the second or more dose of said buccal, lingual or sublingual dosage form comprising epinephrine comprises an amount of epinephrine that is from about 25% to about 200% of the amount of epinephrine in the first dose of said buccal, lingual or sublingual dosage form comprising epinephrine.

43. The method of claim 1, wherein the second or more dose of an injectable dosage form comprising epinephrine comprises an amount of epinephrine greater than the first dose of said injectable dosage form comprising epinephrine.

44. The method of claim 1, wherein the second or more dose of an injectable dosage form comprising epinephrine comprises an amount of epinephrine less than the first dose of said injectable dosage form comprising epinephrine.

45. The method of claim 1, wherein the second or more dose of said injectable dosage form comprising epinephrine comprises an amount of epinephrine that is from about 25% to about 200% of the amount of epinephrine in the first dose of said injectable dosage form comprising epinephrine.

46. The method of claim 1, wherein the time interval between administering each consecutive dose of epinephrine is between about 3 minutes to about 10 minutes.

47. The method of claim 46, wherein each said time interval is about 5 minutes.

48. A kit or packaging system for use in the treatment an allergic emergency in a patient, comprising
a. at least one dose of a buccal, lingual or sublingual dosage form comprising epinephrine; and
b. at least one dose of an injectable dosage form comprising epinephrine.

49. The kit or packaging system of claim 48, further comprising written instructions for administering said at least one dose of a buccal, lingual or sublingual dosage form comprising epinephrine and said at least one dose of an injectable dosage form comprising epinephrine.

50. The kit or packaging system of claim 49, wherein said written instructions provide that:
a. said at least one dose of a buccal, lingual or sublingual dosage form comprising epinephrine is first administered to said patient to provide a therapeutic effect in response to said allergic emergency; and
b. subsequent to said first administration of said at least one dose of a buccal, lingual or sublingual dosage form comprising epinephrine, said at least one dose of an injectable dosage form comprising epinephrine is administered to said patient.

51. The kit or packaging system of claim 49, wherein said written instructions provide that:
a. said at least one dose of an injectable dosage comprising epinephrine is first administered to said patient to provide a therapeutic effect in response to said allergic emergency; and
b. subsequent to said first administration of said at least one dose of an injectable dosage form comprising epinephrine, said at least one dose of an buccal, lingual or sublingual dosage form comprising epinephrine is administered to said patient.

52. The kit or packaging system of claim 48, wherein said at least one dose of a buccal, lingual or sublingual dosage form comprising epinephrine is contained within a protective liner.

53. The kit or packaging system of claim 52, wherein said protective liner prevents damage due to moisture, light, or oxygen.

54. The kit or packaging system of claim 52, wherein said protective liner is a polymer-lined foil.

55. The kit or packaging system of claim 48, wherein said doses of epinephrine are identified in said kit or packaging system.

56. The kit or packaging system of claim 55, wherein said doses of epinephrine are identified by numerical markings or by location within said kit or packaging system.

57. The kit or packaging system of any of claim 55, wherein said identification indicates the order in which the doses are administered to said patient.

58. The kit or packaging system of claim 48, further comprising a carrying case.
